(19) United States
(12) Patent Application Publication
(10) Pub. No.: US 2022/0412947 A1
(43) Pub. Date: Dec. 29, 2022

(54) METHOD AND APPARATUS FOR ELECTROCHEMICAL SCREENING OF CHEMICALS IN THE ENVIRONMENT AND BIOLOGICAL SAMPLES

(71) Applicant: Rutgers, The State University of New Jersey, New Brunswick, NJ (US)

(72) Inventors: Mehdi Javanmard, West Windsor, NJ (US); Robert Miskewitz, Cranford, NJ (US); Ali Maher, Skillman, NJ (US); Clifton Lacy, Highland Park, NJ (US); Azam Gholizadeh, Blacksburg, VA (US)

(73) Assignee: Rutgers, The State University of New Jersey, New Brunswick, NJ (US)

(21) Appl. No.: 17/757,780

(22) PCT Filed: Dec. 16, 2020
(86) PCT No.: PCT/US20/65227

§ 371 Date: Jun. 21, 2022

Related U.S. Application Data
(60) Provisional application No. 62/951,751, filed on Dec. 20, 2019.

(51) Int. Cl. G01N 33/487; G01N 33/18; G01N 27/30; G01N 27/42; G01N 33/24; G01N 33/20

(52) U.S. Cl. CPC G01N 33/48714; G01N 33/1813; G01N 27/308; G01N 27/42; G01N 33/24; G01N 33/20; G01N 2001/4038

(57) ABSTRACT

A sensor apparatus for detecting a heavy metal in a sample.

### CROSS-REFERENCE TO RELATED APPLICATIONS

This application claims priority under 35 U.S.C. § 119(e) to U.S. Provisional Patent Application No. 62/951,751, filed Dec. 20, 2019. The foregoing application is incorporated by reference herein.

### STATEMENT REGARDING FEDERALLY FUNDED RESEARCH

The research leading to the present invention was supported in part, by U.S. Department of Transportation Grant No 69A3551847102. Accordingly, the U.S. Government has certain rights in this invention.

### BACKGROUND

Presence of heavy metals in the environment poses a serious public health and pollution problem. One of the most dominant heavy metals present in the environment is lead as it is widely used in building materials, lead paints, and even lead-acid batteries. As a result of this overabundance in the environment, particularly in natural water sources and even drinking water, lead poisoning has resulted in many public health epidemics. Lead and other toxic metals that end up in natural water sources can often settle down to the bottom into the sediment. Contaminated sediment can get re-suspended into natural water sources due to storms and transit of boats and vessels and may be hazardous to both humans and other life forms. Lead and other toxic metals are known to damage many human organ systems including, but not limited to, the nervous (especially in children), pulmonary, renal, gastrointestinal, hematologic, cardiovascular, and reproductive systems.

Hence, the ability to rapidly screen sediment, water, and other environmental and biological samples for heavy metals such as lead is necessary to identify contaminated areas where remediation is necessary, to thus minimize the risk of exposure and harm. A variety of measurement methods have been developed for heavy metal screening that are primarily based on ion selective electrodes and stripping voltammetry (such as atomic absorption spectroscopy, inductive coupled plasma mass spectroscopy, and various optical and electrochemical methods). While such methods work well in purified buffers within a laboratory, they are not capable of direct measurement of lead and other heavy metals in sediment, water, and other environmental and biological samples. The ability to detect lead and other heavy metals directly in sediment, water, and other environmental samples, as well as in biological samples, with a minimum volume of pretreatment agents and time is needed for in-situ detection of heavy metals for environmental and biological monitoring. Rapid and real time detection of heavy metals in sediment, water, and other environmental and biological samples is very crucial in the field of toxic chemical monitoring. There is a desire for an integrated on-chip sample-to-answer platform capable of detecting lead and other heavy metal ions directly in sediment, water, and other environmental and biological samples at the location of the toxic chemicals (in sediment, water, other environmental sources, and living organisms).

There exists a need for automated, portable, ultra-compact sensing devices capable of qualitatively identifying toxic metals directly in complex biological samples and environmental samples without the need for handling and sample preparation to screen environmental and biological samples for heavy metals. The key challenge to utilizing electrochemical methods for detecting heavy metals in complex matrices such as sediment, food, blood, other body fluids and tissues, and soil at point-of-use is the requirement to perform a separate pretreatment step for extraction of ions and purification of sample. For lead, the ion extraction step is essential to convert all various chemical forms of lead to leads ions (e.g., Pb²⁺) so that they can participate in the electrochemical reaction.

This document describes a novel solution that addresses some of the issues described above.

### SUMMARY

In an embodiment, a sensor apparatus for detecting a heavy metal in a sample includes a reaction vessel, a filter, and a testing chamber. As an example, in one embodiment, the reaction vessel includes a pre-treatment chamber configured to digest the sample to generate ions corresponding to the heavy metal. The testing chamber includes an electrochemical sensor configured to detect the generated ions. The filter allows the generated ions to pass into the testing chamber while blocking other components of the sample. Optionally, the filter may be a cellulose sponge. Optionally, the sample may be a sediment sample. Alternatively, the sample may be an organic sample or an inorganic sample.

As an example, in another embodiment, the electrochemical sensor includes a working gold electrode coated with a graphene oxide film.

As an example, in yet another embodiment, the heavy metal is lead and the generated ions comprise lead ions. Optionally, the pre-treatment chamber may be configured to generate the lead ions in the presence of nitric acid.

As an example, in yet another embodiment, the heavy metal is aluminum, antimony, arsenic, barium, bismuth, cadmium, chromium, cobalt, copper, gold, iron, lithium manganese, mercury, nickel, phosphorus, platinum, selenium, silver, thallium, tin, zinc, in addition to others and the generated ions are comprised therefrom. Optionally, the pre-treatment chamber may be configured to generate the ions in the presence of a reagent for digestion and reaction conditions.

### BRIEF DESCRIPTION OF THE DRAWINGS

FIGS. 1A and 1B are schematic illustrations of example sensor apparatus.

### DETAILED DESCRIPTION

Terminology that is relevant to this disclosure is provided at the end of this detailed description. The illustrations are not to scale.

Rapid and real time detection of heavy metals in sediment, water, and other environmental and biological samples is very crucial in the field of toxic chemical monitoring. The ability to detect heavy metals such as lead and other heavy metals in sediment, water, and other environmental and biological samples with a minimum volume of pretreatment agents and time is needed for in-situ detection of heavy metals for toxic chemical monitoring. This disclosure describes an integrated on-chip sample-to-answer platform capable of detecting heavy metals directly in environmental and biological samples. It should be noted that while the current disclosure describes embodiments for detection of lead, the disclosure is not so limiting, and the systems and methods described herein may be used for detection of heavy metals such as, without limitation, aluminum (Al), antimony (Sb), arsenic (As), barium (Ba), bismuth (Bi), cadmium (Cd), chromium (Cr), cobalt (Co), copper (Cu), gold (Au), iron (Fe), lead (Pb), lithium (Li), manganese (Mn), mercury (Hg), nickel (Ni), phosphorus (P), platinum (Pt), selenium (Se), silver (Ag), thallium (Ti), tin (Sn), zinc (Zn), in addition to others.

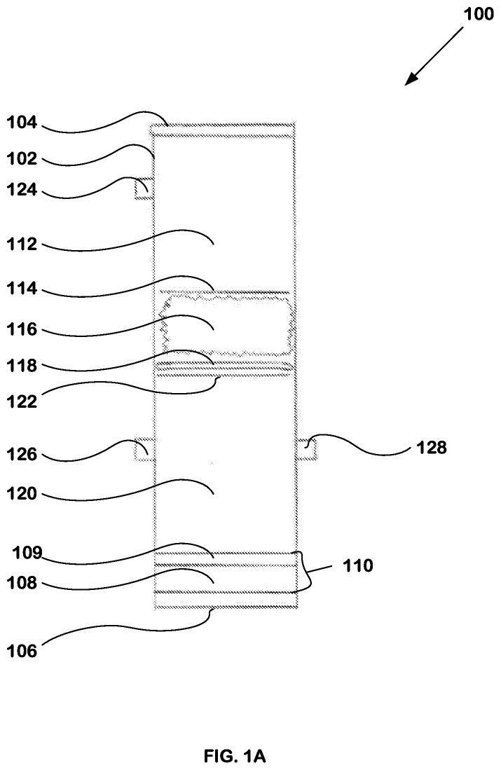

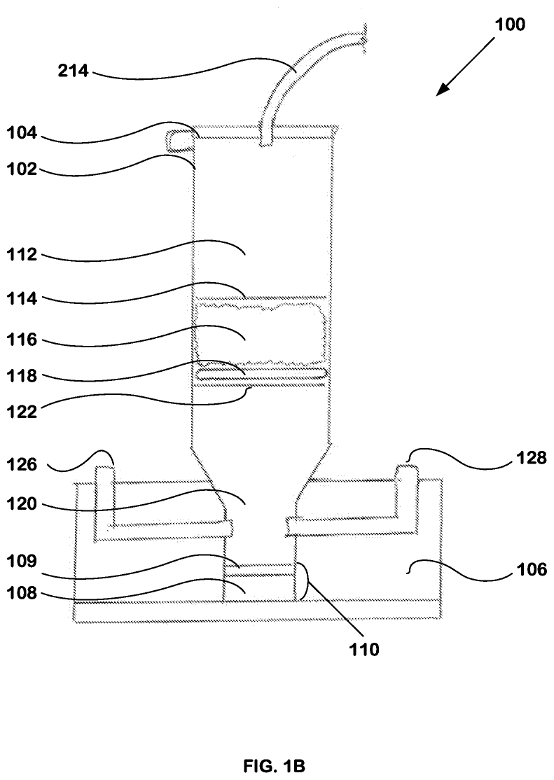

Referring now to FIG. 1A, a schematic illustration of an example sensor apparatus 100 for electrochemical screening of toxic chemicals in a sample is shown. The sensor apparatus 100 may include a reactor vessel 102 with a top opening 104 on one end and a bottom substrate 106 on another end. Optionally, the top opening may include a lid or other type of covering attached to the reaction vessel 102 by a hinge, threads, snap fit, or the like. The size and shape of the reaction vessel 102 may be sufficient to collect a sample for testing. Examples of the sample may include, without limitation, soil, blood, other body fluids (for example, urine, saliva, sputum, tears, semen, milk, vaginal secretions or the like), solid organic tissue (for example, hair, nails, teeth or the like), water, wastewater, food, sewage, sediment sample, or the like. In some embodiments, samples including blood may be collected using either venipuncture or finger prick and sediment samples may be collected using grab or coring samplers.

The reaction vessel 102 include two chambers—a pre-treatment chamber 112 and a testing chamber 120 separated from each by a filter 116.

An untreated sample may be placed within the pre-treatment chamber 112 that is located between the top 104 and the filter 116 for digestion using a suitable reagent such as, without limitation, nitric acid, and other agents (for example, other acids, bases, enzymes, or cellular lysing agents which desorb heavy metals from organic and inorganic substances). The method of digestion may be determined based on, for example, the type of sample and the type of heavy metal being tested. The pre-treatment chamber 112 may include an optional inlet 124 for receiving the reagent to assist in the digestion of the sample. During digestion, the heavy metal to be tested may be converted to corresponding ions for subsequent electrochemical detection. For example, lead in a sediment sample may be converted to Pb²⁺ ions using nitric acid in the pre-treatment chamber 112. The reagent used for digestion and reaction conditions (e.g., the reaction time) may be chosen based on properties of the heavy metal to be detected, the sediment sample, the electrochemical sensor, etc. An optional first membrane 114 may be disposed on top of the filter 116 to prevent large solid particles from entering the testing chamber 120.

The filter 116 may be any suitable porous material that is configured to prevent solid particles from passing into the testing chamber 120, while allowing the toxic metal ions to pass through. The filter 116, therefore, prevents contact between the solid particles and the electrochemical sensor 110. In example embodiments, the filter 116 may be a cellulose sponge. Optionally, the filter 116 may be held in place using an O-ring 118 to prevent fluid (such as reagents) from leaking into the testing chamber 120 without first passing through the filter 116. An optional membrane 122 may be disposed below the filter 116 for additional filtration.

The testing chamber 120 includes an electrochemical sensor 110 fixed to the bottom substrate 106, and allows the filtered sample to contact the electrochemical sensor 110. The bottom substrate 106 may be separate from the reaction vessel 102 or may be integral with the reaction vessel 102. The substrate 106 may be formed from any suitable material capable of providing a suitable support for the electrochemical sensor 110 and the testing chamber 120. For example, the substrate 106 may be formed from a polymer such as polydimethylsiloxane (PDMS), silicones, or the like. The height of the substrate 106 may be about 3 mm-7 mm, about 4 mm-6 mm, about 5 mm, about 4 mm, about 6 mm, or the like.

In certain embodiments, the substrate 106 may have a larger width than that of the testing chamber 120, as shown in FIG. 1B. In such an embodiment, the reaction vessel 102 may be attached to the substrate 106 by, for example, punching a suitable size hole (e.g., about 8 mm) in the substrate 106, adding the electrochemical sensor 110 in the hole, and attaching the reaction vessel 102 in the hole (e.g., via an adhesive). As discussed above, a filter like a cellulose sponge 116 may be located between the untreated sample and the electrochemical sensor.

The electrochemical sensor 110 may be any suitable sensor that is capable of detecting the toxic metal ions of interest accurately and efficiently. Example embodiments may include a working gold electrode 108 coated with a graphene oxide (GO) film 109. Gold electrodes have conductivity and exhibit excellent performance of stripping voltammetry and may be used as working electrode for deposition of GO in an example embodiment). Other working electrodes such as, without limitation, mercury, graphite, glassy carbon, and diamond thin film may also be used. Graphene based nanomaterials coated on electrodes may act as sensitive sensors for heavy metals (e.g., lead ions) due to their extraordinary electronic transport properties, large surface area, higher cathodic window allowing avoidance of reduction of hydrogen, and high electro-catalytic activities. Among them, graphene oxide, prepared through extensive chemical exfoliation of graphite flakes, has oxygen containing functional groups such as hydroxyl, carboxy, epoxy, ether, diol and ketone, which are active sites for adsorbing heavy metals (such as lead). Reduced graphene oxide may also be used.

Various methods may be used for relatively easy deposition of graphene oxide (GO) and/or reduced GO thin films on electrode surfaces including drop casting, dip coating, Langmuir-Blodgett based deposition, transfer via vacuum filtration, and spin coating. The method used for deposition of GO is very important to control surface morphology, film uniformity, thickness and surface coverage. Among these methods, dip coating and drop casting often result in non-uniform deposition due to aggregation of GO sheets. Also, drop casting GO suspension usually results in weak adhesion to electrode substrate. The rapid evaporation of the solvent during spin coating allows a more uniform surface with minimal wrinkling and increases the adhesion between the GO thin film and the electrode surface, which is critical during electrochemical reduction of GO. In example embodiments, a 2 mg mL⁻¹ GO film layer 109 may be placed on the surface of a working gold electrode 108. A 50 μm of a GO large sheet can form a uniform layer in most areas despite the roughness (micron scale features) of the gold electrode surface. From the height profile, the film can be approximated to have a thickness of about 0.5 nm-10 μm that is typical amount for a GO sheet.

The surface morphology and chemical characterization of the GO film on the electrode may be done using scanning electron microscopy (SEM), atomic force microscopy (AFM), Raman Spectroscopy, or the like. The analytical parameters affecting the sensor performance and the thin film fabrication may be described in terms of concentration of GO solution during spin coating, the effect of GO reduction, the supporting electrolyte, square wave anodic stripping voltammetry (SWASV) parameters such as deposition time, frequency, pulse height, or the like. The reliability of the electrode in response to high concentrations of the heavy metal (such as lead) may be also investigated to ensure the capacity of the heavy metal adsorption on the surface of the electrode. Furthermore, the effect of water extracted from the sample on the supporting electrolyte may be investigated both with and without adding acetate buffer solution in the range of 0 ppb to 20 ppm lead standard. This may be then used to quantify the amount of lead present in digested samples collected from environmental samples such as sediment and water, and biological samples.

Additionally and/or alternatively, electrochemical sensors may utilize a variety of electrode surface modifications for increasing sensitivity of metal detection. One example involves the reaction between tin and bismuth with heavy metals such as lead and incorporation of these materials on the surface of the electrodes. Also, a variety of other metal nanoparticles may be used, with the aim of increasing the surface area. Other methods may include use of DNA enzymes.

Because of the ease and low cost for manufacturing, screen printed electrodes (SPE) may also be used as the electrochemical sensor in the sensor apparatus of the current disclosure. Various heavy metal sensors may be formed using SPEs. One example is disposable bismuth oxide modified SPE for detection of lead in the range of 20-300 ppb with a detection limit of 8 ppb. In other works, SPEs modified by gold films displayed very highly linear behavior in the lead concentration range of 2-16 ppb with a detection limit of 0.5 ppb.

The testing chamber 120 may, optionally, include a buffer inlet 126 for receiving a suitable buffer and a buffer outlet 128, as will be described in more details below. An aqueous solution, for example, containing an acetate, PBS, or the like may act as the buffer.

The key challenge to utilizing electrochemical methods for detecting heavy metals in complex matrices such as sediment, food, soil, water, or other environmental or biological samples at point-of-use is the requirement to perform a separate pretreatment step for extraction of ions and purification of sample. The sensor apparatus of the current disclosure solves this problem by providing an ultra-compact sample pretreatment module combined with a highly sensitive electrochemical sensor to detect toxic metal ions in untreated samples obtained directly from water and other environmental and biological sources by separating the untreated sample from the active site of the sensor using a filter such as cellulose sponge. Sponges may adsorb contamination, thus making them a suitable choice of material for the filter. The porous membrane adsorbs contamination of sediment or other contaminants preventing direct contact between solid contaminants like sediment and the active GO site. Moreover, lead ions easily penetrate through the cellulose sponge membrane and thus pure solution reaches the GO surface. An additional benefit to this approach is that this set up minimizes the required acidic pretreatment to microliter levels. The sensor apparatus 100, therefore, is a sample-to-answer platform capable of on-chip contaminant digestion and sample purification in conjunction with electrochemical quantification of metal ions such as lead. Moreover, an electrochemical sensor including a graphene oxide film coated on a gold electrode (e.g., a graphene oxide film selectively spin-coated uniformly onto a screen printed gold working electrode) may provide optimized performance over a wide range of parameters.

As SWASV (Square Wave Anodic Stripping Voltammetry) has proven to be a powerful electrochemical method for sensing heavy metal ions, it may be selected as an example method for detection of metal ions in the sensor apparatus of the current disclosure. The effect of the SWASV on the parameters are most important, namely accumulation potential, time, the number of pulses, and the applied frequency, and may be optimized and calibrated prior to use of the sensor apparatus for detection of heavy metals in samples using any now or hereafter known methods. In certain example embodiments, the sensitivity of the sensor will allow a low detection limit of 0.5 ppb.

In certain embodiments, the design may be modified to incorporate precise microfluidic control to enable automation. To allow sufficient time for contaminant digestion and minimize user handling of reagents, a column may be used to introduce samples containing contaminant interferences and nitric acid to the device. In addition, this design may effectively decrease the volume of nitric acid (which is a hazardous substance) required to the microliter range. Moreover, separate inlet and outlets added to the set up may allow a user to more precise control the ratio of reagents. They may be located as follows: The nitric acid inlet 124 may be located at the top of the reaction vessel 102. The buffer outlet 126 may be located in middle of the PDMS hole. The buffer inlet 128 of the acetate buffer may be located on top of the hole. With this design, reagents are introduced at a (1:1) ratio to help obtain reproducible results comparable to those obtained from calibration experiments.

In certain other embodiments, a closed-feedback loop may also be incorporated based on input set up which allows the sample to be exposed several times to the same volume of pre-treatment reagent, in order to increase the exposure time.

Furthermore, the potentiostat used to readout the electrochemical sensor into a portable instrument may be also be miniaturized resulting in a low cost rapid field analyzer capable of sample-to-answer analysis simultaneously with collection of samples, which can be an alternative to expensive and time-consuming methods such as atomic adsorption and Inductively Coupled Plasma Mass Spectrometry.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.” When used in this document, the term “exemplary” is intended to mean “by way of example” and is not intended to indicate that a particular exemplary item is preferred or required.

When used in this document, terms such as “top” and “bottom,” “upper” and “lower”, or “front” and “rear,” are not intended to have absolute orientations but are instead intended to describe relative positions of various components with respect to each other. For example, a first component may be an “upper” component and a second component may be a “lower” component when a device of which the components are a part is oriented in a first direction. The relative orientations of the components may be reversed, or the components may be on the same plane, if the orientation of the structure that contains the components is changed. The claims are intended to include all orientations of a device containing such components.

In this document, when terms such “first” and “second” are used to modify a noun, such use is simply intended to distinguish one item from another and is not intended to require a sequential order unless specifically stated. The terms “approximately” and “about” when used in connection with a numeric value, is intended to include values that are close to, but not exactly, the number. For example, in some embodiments, the term “about” may include values that are within +/−10 percent of the value.

The above-disclosed features and functions, as well as alternatives, may be combined into many other different systems or applications. Various presently unforeseen or unanticipated alternatives, modifications, variations or improvements may be made by those skilled in the art, each of which is also intended to be encompassed by the disclosed embodiments.

1. A sensor apparatus for detecting a heavy metal in a sample, the sensor apparatus comprising:
a reaction vessel comprising:
a pre-treatment chamber configured to digest the sample to generate ions corresponding to the heavy metal,
a filter, and
a testing chamber comprising an electrochemical sensor configured to detect the generated ions, wherein the filter allows the generated ions to pass into the testing chamber while blocking other components of the sample.

2. The sensor apparatus of claim 1, wherein the filter comprises a cellulose sponge.

3. The sensor apparatus of claim 1, wherein the electrochemical sensor comprises a working electrode coated with a graphene oxide film.

4. The sensor apparatus of claim 1, wherein the heavy metal exemplified is lead but may be any of the heavy metals identified in the Detailed Description and the generated ions exemplified comprise lead ions but would be the ions of the heavy metal of interest.

5. The sensor apparatus of claim 4, wherein the pre-treatment chamber is configured to generate heavy metal ions (lead ions in this example) in the presence of a reagent for digestion and reaction conditions (nitric acid in this example).

6. The sensor apparatus of claim 1, wherein the sample comprises an environmental sample.

7. The sensor apparatus of claim 6, wherein the environmental sample comprises a sediment sample.

8. The sensor apparatus of claim 6, wherein the environmental sample comprises a water sample.

9. The sensor apparatus of claim 1, wherein the sample comprises an organic sample.

10. The sensor apparatus of claim 1, wherein the sample comprises an inorganic sample.

11. The sensor apparatus of claim 1, wherein the sample comprises a biological sample.

12. The sensor apparatus of claim 11, wherein the biological sample comprises a blood sample.

13. The sensor apparatus of claim 11, wherein the biological sample comprises a body fluid sample.

14. The sensor apparatus of claim 11, wherein the biological sample comprises a tissue sample.
